(19) United States
(12) Patent Application Publication
(10) Pub. No.: US 2002/0187165 A1
(43) Pub. Date: Dec. 12, 2002

(54) Composition for female sexual arousal

(75) Inventor: Marie Helena Harbeck, Austin, TX (US)

Correspondence Address: Joseph F. Long, Suite 200, 2508 Ashley Worth Blvd., Austin, TX, 78738, US

(73) Assignee: Joseph F. Long

(21) Appl. No.: 09/854,957

(22) Filed: May 15, 2001

(51) Int. Cl. A61K 31/496; A61K 31/4745; A61K 35/78

(52) U.S. Cl. 424/400; 514/252.16; 514/254.07; 424/757; 514/283; 424/776

(57) ABSTRACT

Compositions for female sexual arousal stimulating vaginal lubrication, vaginal and clitorial engorgement; said compositions contain aphrodisiacs, spermicidal and fungicidal components, may be applied intravaginally, extravaginally, and transdermally, and as a condom lubricant; and said compositions are contained in a water miscible cream base.

### BACKGROUND OF INVENTION

This invention relates to compositions for stimulating vaginal lubrication and sexual arousal in females and for female sexual arousal enhancement, and more particularly to the administration of and treatment of an arousal, spermicidal and fungicidal composition to a human female intravaginally and stimulating vaginal lubrication and sexual arousal composition extravaginally and transdermally.

To the best of the applicant's knowledge the following is the most relevant prior art

| | |
|---|---|
| U.S. Pat. No. 5,945,117 El-Rashidy, et al. | 1999 |
| U.S. Pat. No. 6,031,002 Wysor et al | 2000 |
| U.S. Pat. No. 6,049,240 See | 2000 |
| U.S. Pat. No. 6,051,555 Hadley | 2000 |
| U.S. Pat. No. 6,193,992 El-rashidy, et al | 2001 |
| U.S. Pat. No. 6,194,433 Cutler | 2001 |

Studies have revealed that approximately 10 million women in the United States between the ages of 50 and 74 reported a lack of vaginal lubrication and sexual arousal on 229 million sexual intercourse occasions. In fact, it was found that over fifty-eight percent of 260 females surveyed were affected by sexual dysfunction and lack of vaginal lubrication. as defined as pain or discomfort during sexual intercourse, insufficient vaginal lubrication. delayed or non existent vaginal engorgement, increased time for arousal, diminished ability to reach orgasm, or diminished clitoral sensation.

Female sexual dysfunction increases with age, vascular illness, diabetes, thyroid dysfunction, onset of menopause, and hormonal imbalances and fluctuations.

Female sexual dysfunction has not been addressed sufficiently nor studied as extensively as male sexual dysfunction, due to the historical belief that female sexual dysfunction was related to lack of sexual libido.

There is a growing body of evidence that women with sexual dysfunction commonly have physiologic abnormalities, such as vasculogenic female sexual dysfunction, contributing to their overall sexual health problems. Present day management of women with sexual arousal disorder, especially those with diminished vaginal lubrication and lack of sexual arousal, as well as a lack of vaginal and clitoral engorgement may be impaired by their vasculogenic dysfunction resulting in inadequate blood flow to the clitoral glans, labia minorra and vulval areas.

Most recently, studies with females using the present invention have been shown to have excellent vascular bioavailability. For example, it was found in one study that an intravaginal and extravaginal application of 10.8 mg of Yohimbe incorporated in a water miscible cream formula was optimum for producing a vasculogenic effect on vaginal and clitoral blood flow in a 36 year old female. A significant sexual readiness was evident in the female subject fifteen minutes after application, which produced clitorial erectogenesis, vaginal engorgement, vaginal lubrication, labia minorra engorgement, as well as very aroused sexual responses and readiness for sexual intercourse. During intercourse, the female subject reported that orgasm was faster to attain, whilst the male partner reported that he experienced a very prolonged penile erection due to intravaginal contact with the formulation. Both female and male subjects reported that sexual arousal was still high, three hours after the initial application. Failure or loss of sexual desire to attain orgasm is encountered more frequently in women than in men. Yet, effective, safe, non toxic formulations for treating women who have difficulty in attaining orgasm have not been available to them.

The pharmaceutical industry is expanding its efforts in order to provide treatment for female sexual dysfunction and arousal disorders, thus methods of stimulating dopamine receptors in the mid-brain region of a human female are administered orally and intravenously, as well as toxic, nausea inducing synthetic vasculardilator compositions and methods employed continue to increase yearly.

El-rashidy, et al, U.S. Pat. No. 5,945,117, 1999, has shown a method of ameliorating sexual dysfunction by administering to a female apomorphine, or beta.-cyclodextrin or acceptable acid addition salt as an oral dosage form and in a sufficient amount to increase blood flow to the vaginal wall in amounts less than the amount that induces substantial nausea. This method of stimulating dopamine receptors in the mid-brain region of a human female during sexual activity given orally and administered intravenously in doses in sufficient amount less than induces substantial nausea, has shown to have very poor bioavailability in providing a practical therapeutic use in the areas of female sexual dysfunction.

Wysor, et al, U.S. Pat. No. 6,031,002, 2000, discloses the use of prostaglandin. papaverine, sodium nitroprusside, phenoxybenzamine, phentolamine, prazosin, and vocative neuropathies and their natural and synthetic analogs, dimethylsulfoxide and its analogs, a vasodilator such as alprostadil, a prostaglandin of the E series and its analogs Prostaglandins have been used to treat male impotence and in the treatment of females who exhibit nuerotic symptoms which may cause female sexual dysfunction. The method of topical use with minimum toxicity levels as disclosed in the above mentioned patent dilates the vaginal blood vessels within three to four minutes, has shown that without sufficient time for vaginal lubrication to take place, sexual penetration will be painful for the female subject, even in an aroused state, based on psychologic, hormonal interventions. Antihypertensive drugs, tranquilizers and antidepressants, and many other agents may all cause decreased erectile capacity.

U.S. Pat. No. 6,046,240, 2000, discloses the use of prostanoid compounds, prostaglandin E-1, prostaglandin A, prostaglandin F, including PGF-2, PGF-D, prostacylins, thromboxanes, leukotrienes, 6-keto-PGE-1 derivatives, carbacyclin derivatives, PGD-2 derivatives and the like, and synthetic analogs of any portioned compounds, in a methylcellulose vehicle, and has shown that the doses of portioned compounds should be maintained at relatively low doses due to the potential toxicity of the portioned compounds caused by rapid absorption of the portioned compounds across the vaginal tissue. Antihypertensive drugs, tranquilizers and antidepressants, and many other agents may all cause decreased erectile capacity.

Hadley, U.S. Pat. No. 6,051,555, 2000, discloses the use of peptides from a group consisting of, norleucine, aspartic acid, histidine, d-phenylalanine, arginine, tryptophan, lysine, glycine, proline, tyrosine, and serine. As shown in male human testing, each of the peptides induced an erection in the human male, which was enhanced in combination with a penile injection of 10 mg of peptide per ml of physiological saline solution to produce the erection. As shown, no human female testing has been done, and it is shown that the peptides in this invention may be used in animal husbandry breeding programs.

El-rashidy, et al, U.S. Pat. No. 6,193,992, 2001, discloses apormorphine, hydrochloride salt, .beta.-cyclodextrin, beta.-cyclodextrin is hydroxypropyl-.beta.-cyclodetrin, is administered in a sublingual dose. As shown, in this disclosure, dopamine receptors in the mid-brain region of the human female are used to stimulate the female during sexual activity.

Cutler, U.S. Pat. No. 6,194,433, 2001, discloses treating female sexual dysfunction with oral administrated flosequinan, wherein flosequinan is also administered cutaneously and transurethrally, and contemplates using a variety of quino line derivatives such as methylthio, methylsulphinyl to smooth muscle cells to increase blood flow. It is shown that with oral administration of flosequinan, peak plasma concentrations of flosequinan are observed 1-2 hours following oral administration.

The problem with the use of the compositions of the cited prior art. El-rashidy, U.S. Pat. No. 5,945,117, for treating female sexual dysfunction are their chemical structures which are known to cause to cause toxicity and substantial nausea, if not administered under strict supervisory conditions.

Unfortunately, in the compositions of the prior art the daily plasma concentrations of actives must be maintained via daily oral adminstration within the plasma of female subjects relating to the test subjects body weight.

The cited prior art compositions, and Cutler, U.S. Pat. No. 6,194,433, do not employ an organic, water-based emulsion as the vehicle for delivering organic vaginal engorgement additives to stimulate female sexual enhancement and arousal.

The prior art compositions that we have found do not employ the use of an organic topical additive as a non toxic female arousal vehicle.

None of the prior art compositions, El-rashidy, et al, U.S. Pat. No. 5,945,117, Wysor, U.S. Pat. No. 6,031,002, U.S. Pat. No. 6,046,240, Hadley, U.S. Pat. No. 6,051,555, El-rashidy, et al, U.S. Pat. No. 6,193,992, employ a spermicidal additive in their female sexual enhancement and arousal compositions.

We found no prior art compositions that employed a spermicidal additive or a fungicidal additive in their female sexual enhancement and arousal compositions.

In terms of effectiveness, we found no prior art compositions that provide satisfactory skin penetration and supplying properties for female sexual arousal within six to ten minutes of applying said composition.

We found no prior art that claimed a composition to be used as a condom lubricant to enhance female sexual arousal.

We found no prior art compositions to provide a fungicidal additive with female arousal enhancement.

We found no prior art compositions to treat female sexual dysfunctions intravaginally.

We found no prior art compositions that claimed to have been tested extensively on human subjects as the present invention has

El-rashidy, et al, U.S. Pat. No. 6,193,992, uses apomorphine which is a selective dopamine receptor agonist that has been widely utilized as an emetic agent, sedative, antiparkinsonian agent and a behaviour altering agent, the effect of apormorphine on human female sexual functionality have not been previously investigated, El-rashidy, et al, U.S. Pat. No. 6,193,992, utilizes the administration of an anti emetic agent that is used in conjunction with apormorphine oral administration to prevent nausea.

Wysor, et al, U.S. Pat. No. 6,031,002, employs prostoglandins in a crosslinked hydroxyl group which is insoluble in water based formulations and would not be rapidly absorbed.

We found no prior art compositions that claimed to be absorbed into the tissues within 39 seconds of application.

We found no prior art compositions that claimed a near neutral composition as in our compositions which have a pH of about 6.9.

Some prior art compositions, See U.S. Pat. No. 6,046,240, use egg yolk phospholipids and soybean phospholipids with their formulations; these are known to cause allergic reactions in some human females.

We found no prior art compositions that use formulations whereby the sexual arousal state presents itself within the female within six minutes of topical application.

We found that composition of this invention can be safely reapplied as often as needed for individual sexual response requirements.

Objectives and advantages of the present invention include:

a) to provide compositions for treating female sexual dysfunction without toxic formulations that are known to cause nausea,

b) to provide formulations whereby daily plasma concentrations of actives do not need to be maintained via daily oral administration,

c) to provide a water-based emulsion as the vehicle for delivering additives to stimulate female sexual enhancement and arousal,

d) to provide a topical additive that is a non-toxic female arousal vehicle,

e) to provide for use of a spermicidal additive in the female sexual enhancement and arousal composition,

e) to provide for the use of a fungicidal vehicle within the female sexual enhancement and arousal formulation,

f) to provide for the use of both a fungicidal and spermicidal vehicle within the female sexual enhancement and arousal formulation,

g) to provide satisfactory skin penetration and supplying properties that provide female sexual arousal within six to ten minutes of applying said composition,

h) to provide a female sexual arousal additive, plus a spermicidal additive and a fungicidal additive that may be applied in an intravaginally formulation;

i) to provide an additive that can be used as a condom lubricant to enhance female sexual arousal;

j) to provide a female sexual enhancement and arousal formulation that has been tested on female subjects,

k) to provide female sexual enhancement and arousal compositions that are free of emetic agents, sedative, antiparkinsonian agents and behaviour altering agents;

l) to provide female sexual enhancement and arousal compositions in a water based, formulation for swift tissue penetration,

q) to provide female sexual enhancement and arousal formulations that are rapidly absorbed,

r) to provide female sexual enhancement and arousal formulations for intravaginal and extravaginal application,

s) to provide non tissue aggravating components within the female sexual enhancement and arousal formulations,

t) to provide formulations for female sexual enhancement and arousal whereby the sexual arousal state presents itself within the human female within a few minutes of topical application,

u) to provide organic formulations whereby the sexual arousal state of the female lasts for 2 to 3½ after topical administration,

v) to provide female sexual arousal formulations that can be safely reapplied as often as needed for individual sexual response requirements,

w) to provide a female sexual arousal formulation that can incorporate within its components fragrant plant oils for the users pleasure,

x) to provide a formulation for female sexual arousal that can be used to lubricate condoms,

y) to provide a formulation for female sexual arousal that can be used to lubricate condoms with a composition comprising a female sexual arousal component, and a spermicidal and fungicidal component.

An urgent need exists in the field of human female sexual dysfunction and arousal enhancement for organic additives that will be readily accepted by the human female body and be without undesirable side-effects, to enhance human female sexual arousal, increase nerve stimulation and blood flow for enhanced clitoral erection, and vaginal lubrication, as well as vaginal engorgement in a human female. Further advantages of this invention are the employment of a near neutral 6.9 pH water-based formulation that starts to penetrate the tissues within 39 seconds of application. Still further objects and advantages will become apparent from a consideration of the ensuing descriptions.

### SUMMARY OF THE INVENTION

In accordance with the present invention a human female sexual arousal enhancement composition for stimulating vaginal lubrication, and vaginal and clitoral engorgement, that includes one or more aphrodisiac agents and spermicidal and fungicidal vehicles and is for use intravaginally, extravaginally, intracervical, transdermally, and as a condom lubricant in a water miscible cream base comprising water, cocoa butter, pro lipo H166.0, glycerol monostearate, carbomer, jojoba oil, and sodium behenoyl lactylate components.

### DESCRIPTION OF THE INVENTION

The water miscible cream base comprises:

| | |
|---|---|
| 77.0 grams | water, |
| 1.0 grams | cocoa butter, |
| 2.0 grams | pro lipo H166.01 purchased from Lucas Meyer Beauty- |
| | Essentials, E.A.E. LaTuilerie. F77500 Chelles. France, |
| 2.0 grams | glycerol monostearate, |
| 2.0 grams | acritamer carbomer purchased from R.I.T.A Corporation, |
| | 1725 Kilkenny Ct, Woodstock, IL, 60098, |
| 1.0 gram | jojoba oil, |
| 2.0 grams | sodium behenoyl lactylate. |

compounded as follows:

place water and acritamer carbomer in a partially submerged container over hot water to form a mixture, vigorously stirring said mixture over hot water until dissolved;

simultaneously placing cocoa butter, pro lipo H166.01, glycerol monostearate, jojoba oil, and sodium behenoyl lactylate, in another partially submerged container over hot water to form a mixture; stirring said mixture over hot water until dissolved,

removing said mixtures from heat and blending both said mixtures with stirring until said second mixture cools, whereby the ingredients are formed into a cream base.

Extravaginal cream number 1 comprises

2000 parts of cream base,

50 parts Viagara TM.

Intravaginal cream number 2 comprises:

5000 parts cream base,

50 parts Viagra TM.

Extravaginal cream number 3 comprises:

2000 parts cream base,

10.8 parts yohimbe.

Intravaginal cream number 4 comprises:

5000 parts cream base,

10.8 parts yohimbe.

Intravaginal cream number 5 comprises:

5000 parts cream base,

10.8 parts yohimbe.

100 parts nonoxynol 9,

Intravaginal cream number 6 comprises:

5000 parts cream base,

10.8 parts yohimbe,

50 parts clotrimazole.

Intravaginal cream number 7 comprises:

5000 parts cream base,

10.8 parts yohimbe,

100 parts nonoxynol 9,

50 parts clotrimazole.

Extravaginal cream number 8 comprises

2000 parts cream base,

25 parts Viagara TM.

Intravaginal cream number 9 comprises:

5000 parts of cream base,

25 parts Viagara TM,

50 parts clotrimazole.

Intravaginal cream number 10 comprises:

5000 parts of cream base,

25 parts Viagara TM,

100 parts nonoxynol 9.

Intravaginal cream number 11 comprises:

5000 parts of cream base,

25 parts Viagara TM,

100 parts nonoxynol 9,

50 parts clotrimazole.

Intravaginal cream number 12 comprises:

5000 parts of cream base,

25 parts Viagara TM.

Intravaginal cream number 13 comprises:

5000 parts of cream base,

50 parts of Viagara TM.

10.8 parts yohimbe.

Extravaginal cream number 14 comprises

2000 parts of cream base,

50 mg parts Viagara TM,

10.8 parts yohimbe.

Intravaginal cream number 15 comprises:

5000 parts of cream base,

50 parts Viagara TM,

10.8 parts yohimbe,

100 parts nonoxynol 9.

Intravaginal cream number 16 comprises:

5000 parts cream base,

50 parts Viagara TM,

10.8 parts yohimbe,

50 parts clotrimazole.

Intravaginal cream number 17 comprises:

5000 parts of cream base,

50 parts Viagara TM,

10.8 parts yohimbe,

50 parts clotrimazole,

200 parts nonoxynol 9.

All the above compositions may be incorporated in said cream base by finely grinding any solid additives and mixing by stirring in a well known manner.

We have listed several compositions as examples but much stronger aphrodisiac mixtures may be necessary for some individuals. We have also listed only two aphrodisiacs but others such as ginseng and Damiana may work equally well and would be within the purview of the patent. We therefor do not wish to be limited to exact amounts but only to the spirit and purposes as outlined in these claims and specifications.

The suggested and proven method of use is for a female to intervaginally introduce about five millileters of one of the suggested intervaginal mixtures and digitally apply about two millileters in a thin layer to the female genitalia of one of either the extravaginal or intervaginal compositions.

What is claimed is:

1) compositions for female sexual arousal comprising:
a) a water miscible cream base,
b) an aphrodisiac mixed in said cream base;

2) Compositions as in claim 1 wherein said cream base comprises a mixture of approximately 80 parts water, 1 part cocoa butter, 2 parts Pro Lipo HI 166.01, 2 parts glycerol monostearate, 2 parts acritamer carbomer, 1 part jojoba oil and 2 parts sodium behenoyl lactylate.

3 ) Compositions as in claim 1 wherein said aphrodisiac is Yohimbe.

4) Compositions as in claim 1 wherein said aphrodisiac is Viagra TM.

5) Compositions as in claim 1 further comprising a fungicidal component.

6) Compositions as in claim 5 wherein said fungicidal component is Clotrimazole.

7) Compositions as in claim 1 further comprising a spermacidal component.

8) Compositions as in claim 7 wherein said spermicidal component is Nonoxynol 9.

9) A Composition for female sexual arousal as in claim 1 for extravaginal use comprising two thousand parts of said cream base and fifty parts Viagra TM.

10) A composition for female sexual arousal as in claim 1 for intravaginal use comprising five thousand parts of said cream base and fifty parts Viagra TM.

11) A composition for female sexual arousal as in claim 1 for extravaginal use comprising two thousand parts of said cream base and approximately eleven parts Yohimbe.

12) A composition for female sexual arousal as in claim 1 for intravaginal use comprising five thousand parts of said cream base and approximately eleven parts of Yohimbe.

13) A composition for female sexual arousal as in claim 12 for intravaginal use further comprising one hundred parts of Nonoxynol 9, a spermicidal.

14) A composition for female sexual arousal as in claim 12 for intravaginal use further comprising fifty parts Clotrimazole, a fungicidal.

15) A Composition for female sexual arousal as in claim 10 for intravaginal use further comprising one hundred parts Nonoxynol 9, a spermicidal.

16) A composition for female sexual arousal as in claim 10 for intravaginal use further comprising fifty parts Clotrimazole, a fungicidal.

17) A composition for female sexual arousal as in claim 9 for extravaginal use further comprising one hundred parts Nonoxynol 9, a spermicidal.

18) A composition for lubricating a condom comprising two thousand parts of a water soluble cream base, fifty parts Viagra TM, and one hundred parts of Nonoxynol 9, a spermicidal.

19) A composition for lubricating a condom comprising two thousand parts of a water soluble cream base, eleven parts Yohimbe, and one hundred parts of Nonoxynol 9.

20) A method of use by a female of a cream based arousal composition for intravaginal use wherein said female using an applicator inserts vaginally a minimum of about five milliliters of said cream based composition.

21) A method of use by a female of a cream based arousal composition as in claim 19 further comprising applying a thin layer of said arousal composition to genitalia of said female.
